(19) United States
(12) Patent Application Publication
(10) Pub. No.: US 2026/0351345 A1
(43) Pub. Date: Nov. 5, 2026

(54) SYSTEMS AND METHODS FOR PRIMER SELECTION AND MULTIPLEX PCR

(71) Applicant: NUCLEIX LTD., Rehovot (IL)

(72) Inventors: Michael Edward DODGE, San Diego, CA (US); Blake PRINTY, Austin, TX (US); Eran BRAM, San Diego, CA (US)

(73) Assignee: NUCLEIX LTD., Rehovot (IL)

(21) Appl. No.: 19/125,444

(22) PCT Filed: Oct. 31, 2023
(86) PCT No.: PCT/IL2023/051120

§ 371 Date: Apr. 29, 2025

Related U.S. Application Data
(60) Provisional application No. 63/421,145, filed on Oct. 31, 2022.

(51) Int. Cl. G16B 25/20; C12Q 1/6811; C12Q 1/686

(52) U.S. Cl. CPC G16B 25/20; C12Q 1/6811; C12Q 1/686

(57) ABSTRACT

An interlaced (in silico/in vitro) pipeline for rapid and efficient selection of primer sets for use in multiplex PCR reactions; reagents comprising primer sets for use in multiplex PCR reactions identified by the interlaced pipeline, and PCR reaction systems to amplify a plurality of target DNA templates in a multiplexed fashion using the primer sets identified by the interlaced pipeline.

### FIELD OF THE INVENTION

The present invention relates to reagent design for multiplex PCR.

### BACKGROUND OF THE INVENTION

Multiplex PCR is the simultaneous amplification of more than one target sequence in a single reaction tube. Since its introduction, multiplex PCR has been successfully applied in many areas of nucleic acid diagnostics, including gene deletion analysis, mutation and polymorphism analysis, RNA detection, and identification of infectious disease agents. Because each amplification in the multiplex proceeds independently of the others, multiplexing necessarily requires the use of multiple primer sets that are compatible with one another.

The optimization of multiplex PCR reactions poses several challenges. For example, as the presence of different primer pairs in the reaction increases the chance of obtaining spurious amplification products (e.g., through the formation of primer dimers). These spurious products may be amplified more efficiently than the desired target, consuming reaction components and producing impaired rates of annealing and extension. Special attention to primer design parameters such as homology of primers with their target nucleic acid sequences, internal homology within the primer and between primers, primer length, the GC content, melting temperatures (Tm) and their concentration should be taken. Ideally, the goal is that all primer pairs in a multiplex PCR should enable similar amplification efficiencies for their respective target.

Among the processes that induce bias errors into a multiplex PCR reaction include PCR drift (a fluctuation in the interactions of PCR reagents or environmental control, particularly in the early cycles) and PCR selection (unequal amplification of certain templates due to the properties of the target, the target's flanking sequences, or the entire target genome such as regional differences in GC content, higher primer binding efficiency, secondary structure in the template; and the gene copy number). Amplification bias errors may thus be strongly dependent on the choice of primers.

### BRIEF DESCRIPTION OF THE INVENTION

It is an object of the present invention to provide an interlaced (in silico/in vitro) pipeline for rapid and efficient selection of primer sets for use in multiplex PCR reactions. It is a further object of the invention to provide a reagent comprising primer sets for use in multiplex PCR reactions identified by the interlaced pipeline of the present invention. It is a further object of the invention to provide a PCR reaction system to amplify a plurality of target DNA templates in a multiplexed fashion using the primer sets identified by the interlaced pipeline of the present invention.

As described hereinafter, the present invention can proceed from a large set of target DNA templates numbering in the hundreds to a set of validated amplification primer pairs that are internally compatible and exhibit low off-target noise within as little 2-4 weeks. The methods begin by identifying in silico a set of potential “test” primer pairs for each DNA template, filtering them in a first in silico stage to a reduced set of initial primer pairs; further filtering these in a defined set of in vitro amplification reactions to a reduced set of intermediate and then candidate primer pairs, and then returning to an in silico analysis to identify the most compatible set of final primer pairs for the large set of target DNA templates. Finally, a “dark cycling” reaction is used to rapidly confirm multiplex compatibility.

In a first aspect, present invention provides a method for primer selection for a multiplex PCR reaction of a plurality of DNA templates, comprising:
(a) for each DNA template in the plurality of DNA templates, performing an in silico identification of a plurality of test primer pairs, and for each test primer pair calculating in silico pair a first scoring function value based on a set of predetermined selection criteria that comprise one or more of, and preferably each of
   (i) a calculated ranking derived from a calculated melting point (Tm) range, a calculated primer length range, a calculated primer GC content and/or location value, and a calculated amplicon length,
   (ii) a calculated number of predicted primer dimers within the test primer pair and between the test primer pair a control primer pair configured to amplify a predetermined control DNA template to be included in the multiplex PCR reaction,
   (iii) a calculated overlap with a region of the DNA template that is a repeat of 10 bases or longer, and
   (iv) a calculated number of off-target amplification products from a reference genome, and for each DNA template in the plurality of DNA templates, using the first scoring function value to select from the plurality of test primer pairs a plurality of initial primer pairs;
(b) for each template in the plurality of DNA templates, screening each primer pair in the plurality of initial primer pairs in vitro in a single plex real time PCR reaction, calculating for each initial primer pair a second scoring function value based on set of selection criteria that comprise one or more of, and preferably each of, a cycle threshold (Ct) value, a melt curve peak threshold, a secondary melt curve peak, and a ratio of on-target to off-target amplicons produced, and for each DNA template in the plurality of DNA templates, using the second scoring function value to select from the plurality of initial primer pairs a plurality of intermediate primer pairs;
(c) for each template in the plurality of DNA templates, screening each primer pair in the plurality of intermediate primer pairs in vitro in a duplex real time PCR reaction with the control DNA template selected to be included in the multiplex PCR reaction and the control primer pair, and calculating for each intermediate primer pair a third scoring function value based on set of selection criteria that comprise one or more of, and preferably each of, a second Ct value and a peak signal at the end of the duplex real time PCR reaction, and for each DNA template in the plurality of DNA templates, using the third scoring function value to select from the plurality of intermediate primer pairs a plurality of candidate primer pairs;
(d) for each DNA template in the plurality of DNA templates, combinatorially binning the plurality of candidate primer pairs for each DNA template in silico to form a first primer multiplex and for each candidate primer pair calculating in silico pair a fourth scoring function value based on a set of predetermined selection criteria that comprise one or more of, and preferably each of
   (i) a calculated ranking derived from a calculated melting point (Tm) range, a calculated primer length range, a calculated primer GC content and/or location value, and a calculated amplicon length,
   (ii) a calculated number of predicted primer dimers within the candidate primer pair, between the candidate primer pair the control primer pair, and between the candidate primer pair all other primer pairs in the first primer multiplex,
   (iii) a calculated overlap with a template region that is a repeat of 10 bases or longer,
   (iv) a calculated number of off-target amplification products from a reference genome, and
and for each DNA template in the plurality of DNA templates, using the fourth scoring function value to select from the plurality of candidate primer pairs a plurality of final primer pairs;
(e) binning the final primer pair for each template in the plurality of DNA templates to form a set of multiplex primers; and for each template in the plurality of DNA templates, dark cycling the final primer pair in vitro to obtain a final Ct value, wherein the final primer pair is determined to be functional based on comparison of the final Ct value and peak signal obtained from the dark cycling of the final primer pair to the second Ct value and peak signal obtained in (c) for the final primer pair; and
(f) providing a pooled set of final primer pairs for the plurality of DNA templates.

The term “in silico” as used herein with regard to certain steps in the multiplex primer workflow refers to a process performed via computer computation. For example, electronic PCR (ePCR) is a process similar to ordinary PCR, but performed using nucleic acid sequences and primer pair sequences stored in computer-formatted media. In silico PCR primer design and ranking using tools such as PRIMER 3 (Koressaar and Remm, Bioinformatics 2007; 23(10): 1289-1291) MRPrimer (Kim et al., Nucleic Acids Research, 2015 1 doi: 10.1093/nar/gkv632) and GPrimer (Bae et al. BMC Bioinformatics (2021) 22:220 doi: 10.1186/s 12859-021-04133-4) are known in the art. By contrast, the term “in vitro” as used herein as used herein with regard to certain steps in the multiplex primer workflow refers to a laboratory process that is performed by traditional wet chemistry methods.

The term “single plex” as used herein with regard to a PCR reaction refers to a PCR reaction in which a single nucleic acid target is amplified using a single pair of primers.

Similarly, “duplex” as used herein with regard to a PCR reaction refers to a PCR reaction in which two nucleic acid targets are amplified in a single reaction mixture using a different pair of primers for each target.

A “multiplex” as used herein with regard to a PCR reaction refers to a PCR reaction in which more than two nucleic acid targets are amplified in a single reaction mixture using a different pair of primers for each target. A multiplex may include from 3 to many hundreds of individual unique DNA targets. In various embodiments, the methods described herein are used to provide primer pairs for at least 3, 5, 7, 10, 15, 20, 30, 50, 100, 200, 500, or more unique DNA targets.

The term “DNA template” as used herein refers to a nucleic acid sequence or molecule that is intended to be amplified by a pair of primers. “Template” and “target” may be used interchangeably in this context. “Off-target” refers to a nucleic acid sequence other than the intended template that is amplified inadvertently in a PCR reaction. Such off-target sites are not identical to the intended template, but contain sufficient homology to one or more primers present in the reaction to initiate priming.

The term “binning” as used herein for an in vitro system refers to combining primer pairs for more than one nucleic acid target in a single reagent to create a “primer pool.” The term “combinatorially binning” as used herein for an in silico system refers to producing all possible combinations of primer pairs across groups of primer sets. By way of example, if there are three unique DNA targets and each has three associated primer sets, there are 3³=27 possible unique combinations in which the primer pairs may be binned that contain a primer pair for all three unique DNA targets.

The term “dark cycling” as used herein refers to a multiplex PCR method in which only a small number (e.g., one, two, three, etc.) of the amplicons of the multiple unique DNA targets are being detectably labeled, but all of the multiple unique DNA targets are being amplified. This provides a method of determining if a particular primer pair is compatible with the multiple primer pairs required for the multiplex without requiring a different label for each target's amplicons, thus reducing procurement time needed to confirm the viability of the multiplex. Normally, multiplexing several targets on the same color would be impossible in conventional qPCR, as an operator cannot decern which target produced the signal. To bypass this limitation, dark cycling places each target probe into separate wells, while retaining all primers for all targets. This allows each probe to generate signal in each well, while primers for probes that are absent amplify as they normally would but fail to produce signal (so “dark cycling”). These “dark” primers can contribute to off-targeting of the measured signal, and by keeping them present/active, off-targeting and interference between primers can be measured without a true full color multiplex.

In exemplary embodiments described below, beginning with an exemplary 100 unique DNA targets, one begin with all possible primer pairs for each target, and quickly filter these potential primer pairs to a final multiplex set by filtering to 8 initial primer pairs per target at the first in silico stage, 4 intermediate primer pairs per target at the first in vitro stage, 2 candidate primer pairs per target at the first in vitro stage, and arrive at, and validate, the preferred primer pair for the multiplex in a second in silico stage followed by dark cycling.

At various points in this filtration process, the primer pairs being evaluated for a DNA target are evaluated by a scoring function. By way of example, a scoring function may be calculated after the first in silico stage, calculated according to the following equation, and the some number of primer pairs passed to the next stage of the process, with the rest discarded:

Sₚₒₒₗ=Wₒ* N_{offtarget}+W_{d}*N_{dimer}+Wₕ*nₕₐᵢᵣₚᵢₙ+WᵣΣCᵣₐₙₖ,

wherein

N_{offtarget} is the number of predicted off-target products, N_{dimer} is the number of predicted dimer products, Nₕₐᵢᵣₚᵢₙ is the number of predicted hairpin products, Cᵣₐₙₖ is the calculated ranking in (a)(i), Wₒ is the relative weight applied to N_{offtarget}, W_{d} is the relative weight applied to N_{dimer}, Wₕ is the relative weight applied to Nₕₐᵢᵣₚᵢₙ, and Wᵣ is the relative weight applied to Cᵣₐₙₖ.

In an exemplary embodiment described below, Wₒ is set at 1, W_{d} is set at 1, Wₕ is set at 0.25, and Wᵣ is set at 0.5. This would weight off-targeting higher than hairpin formation in the final decision regarding suitable primer sets. These various weightings may be adjusted according to the operator.

The second scoring function is calculated following a first in vitro stage, which is a single plex real time PCR reaction selected for its ability to be run quickly and inexpensively. All that is needed are the primers, which can typically be ordered and tested within 24 hours. No expensive/slow-to-make probes are required. The screening template is typically a normal genome, e.g., obtained from commercially available buffy coat DNA. An exemplary second scoring function based on Ct and Tm peak values is described hereinafter. In certain embodiments, this score can be modified by a check for off-target amplification by direct sizing of the amplicons produced.

The exemplary third scoring function is calculated following a duplex amplification in which the second member of the duplex is a control template intended to be used in the final multiplex in order to remove primer pairs that are incompatible with this control. In exemplary embodiments, the third scoring function is calculated by sorting each primer pair for a particular DNA template at a first level according to the Ct value obtained (lowest is better), and then sorting at a second level by peak signal height at a predetermined cycle number. This second level may be used to select between otherwise identical (Ct value) primers.

In the initial in silico stage, primer pairs are not selected for multiplex compatibility, as the large number of primers available at this stage would be computationally expensive. Thus, a second in silico stage considers the best 1-2 primers per locus (for all needed targets) as identified in the preceding stages. New primer pairs are not generated; rather the remaining primer pairs are checked against each other. A score is calculated in a similar manner as in the first stage, provided that the number of predicted dimer products differs since all primer pairs in the multiplex are considered together:

Sₚₒₒₗ=Wₒ*N_{offtarget}+W_{d}+N_{dimer}+W_{d}*Nₕₐᵢᵣₚᵢₙ+Wᵣ* ΣCᵣₐₙₖ,

wherein

N_{offtarget} is the number of predicted offtarget products, N_{dimer} is the number of predicted dimer products, Nₕₐᵢᵣₚᵢₙ is the number of predicted hairpin products, Cᵣₐₙₖ is the calculated ranking in (d)(i), Wₒ is the relative weight applied to N_{offtarget}, W_{d} is the relative weight applied to N_{dimer}, Wₕ is the relative weight applied to Nₕₐᵢᵣₚᵢₙ, and Wᵣ is the relative weight applied to Cᵣₐₙₖ.

In the dark cycling stage, the final primer pair may be identified as functional when the final Ct value and the peak signal obtained from the dark cycling of the final primer pair is compares favorably to that obtained in the duplex PCR stage described above. This indicates that the performance of the primer pair is not being substantially affected by the other members of the primer multiplex.

In a related aspect, present invention provides a method for performing a multiplex PCR reaction of a plurality of DNA templates, comprising:
obtaining a nucleic acid sample to be amplified;
preparing a PCR reaction mixture comprising the nucleic acid sample and the pooled set of final primer pairs for the plurality of DNA templates according to the methods provided herein; and
incubating the PCR reaction mixture under conditions sufficient to amplify the plurality of DNA templates present in the nucleic acid sample.

In certain embodiments, the nucleic acid sample comprises or consists of cell free DNA (cfDNA). In other embodiments, the nucleic acid sample comprises or consists of genomic DNA (gDNA).

### BRIEF DESCRIPTION OF THE FIGURES

FIG. 1 depicts a flow diagram of an interlaced multiplex primer design workflow according to the invention.

FIG. 2 depicts in vitro screening results of initial PrimerX primer pair outputs using EvaGreen qPCR.

FIG. 3 depicts in vitro fragment analysis results of amplicons generated from initial PrimerX primer pairs in single plex assay (left panel) and full color quad plex assay.

FIG. 4 depicts qPCR results using “clean” vs. “dirty” multiplex primer pair groups.

FIG. 5 depicts a schematic of a dark cycling protocol.

FIG. 6 depicts qPCR results comparing dark cycling of a set of primer pairs (left panel) to a full color multiplex of the same primer pairs (right panel).

FIG. 7 depicts a flow chart showing NGS sequencing and interaction determination of primer pairs iterating with the interlaced protocol of the invention.

FIG. 8 depicts a correlation between off-target results predicted by the PrimerX software and actual off-target results determined by NGS of amplicons in a 30-plex assay.

FIG. 9 depicts a flow diagram of the PrimerX software approach.

FIG. 10 depicts a flow diagram of the PrimerX in silico primer candidate design.

FIG. 11 depicts a flow diagram of the PrimerX in silico primer candidate PCR analysis.

FIG. 12 depicts a flow diagram of the PrimerX in silico primer optimization analysis.

### DETAILED DESCRIPTION OF THE INVENTION

The present invention relates to an interlaced (in-silico/in-vitro) pipeline for rapid and efficient generation of primer pairs compatible with multiplexed PCR amplification-based assays. While described hereinafter with regard to methylation biomarkers for quantification of epigenetic changes in cfDNA from blood, the methods and compositions described herein are generally applicable to any assay system involving multiplexed PCR.

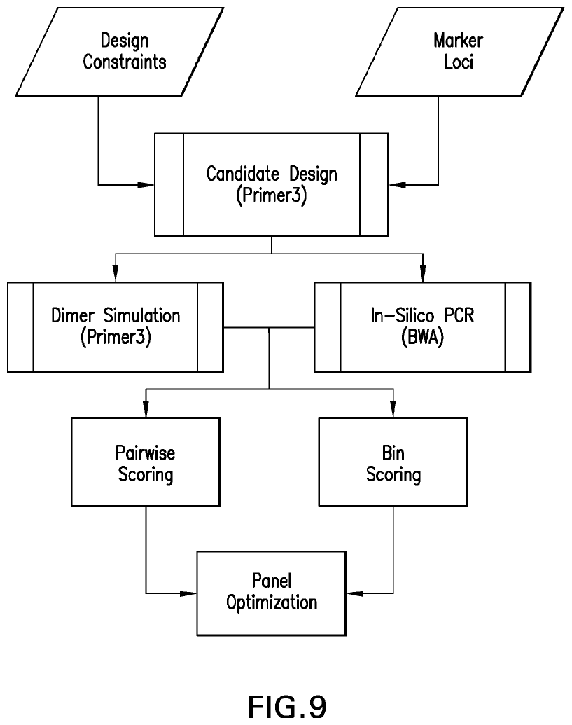

First in silico stage—the PrimerX software PrimerX utilizes a multi-stage workflow for designing optimal primer pairs and probes. Primer candidates are first generated for each marker by the software and subsequently selected into a final design by minimizing unwanted interactions (FIG. 9). From this information, PrimerX formulates design inputs for a PRIMER3 call in several steps. First, thermodynamic parameters are parsed, and sequences are retrieved for genomic loci from an included HG38 (GRCh38 Genome Reference Consortium Human Build 38) reference. Next, a database with 1000 Genomes Project annotation (www.nature.com/articles/526052a) is queried for any sites with a MAF alignment greater than a user-specified threshold (default: 0.05). These sites are excluded from the design as non-viable primer binding sites. After that, repetitive sequences of a user-specified size (default: 12) are detected in marker loci sequences and excluded from the design. Finally, user-specified motifs (e.g. enzyme cut sites) are detected in marker loci sequences and noted in design configuration as special regions to flank with primer pairs.

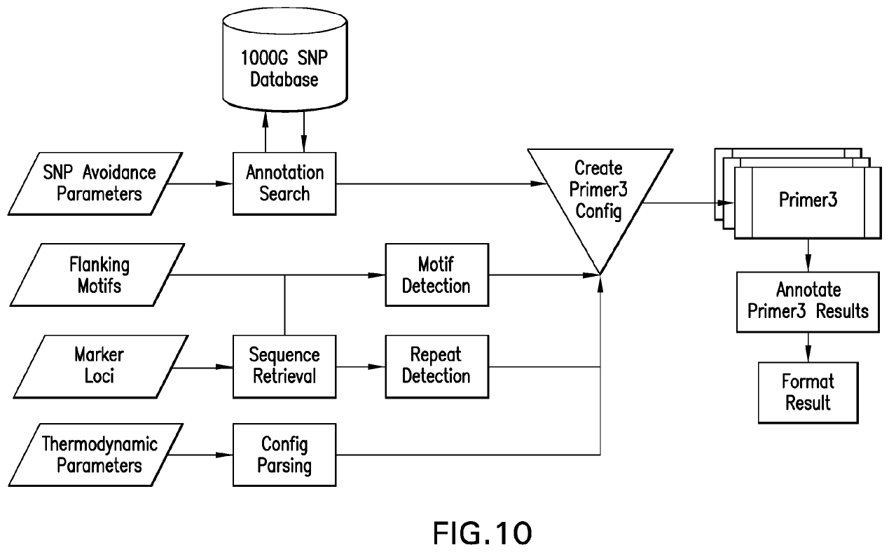

From this information, PrimerX formulates a PRIMER3-compatible design configuration and utilizes PRIMER3 in parallel threads to generate a user-specified number of primer (and probe) candidates (default: 5) for each marker loci. Design results from Primer3 are then aggregated and special motifs annotated for downstream steps. The workflow is shown in FIG. 10.

After candidate primers are generated for each unique marker, an in silico PCR step is performed to predict potential off-targeting from each pairwise interaction possible in the design. The in silico PCR step has four main components. Frist, candidate sequences are aligned to the HG38 reference genome using BWA to verify that expected amplification products are produced by candidate designs. Next, each primer is aligned with BWA using specialized parameters to produce many supplementary alignments with a small burrows-wheeler seed length (default: 10). Exemplary parameters used for individual primer alignments may be as follows:

−y5000−c50000−a−p−s−r1−t0−k 10

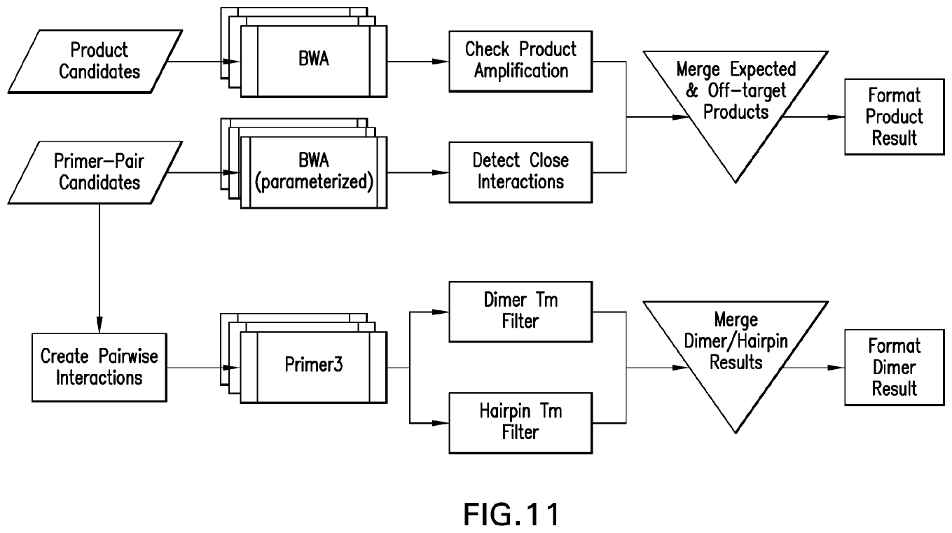

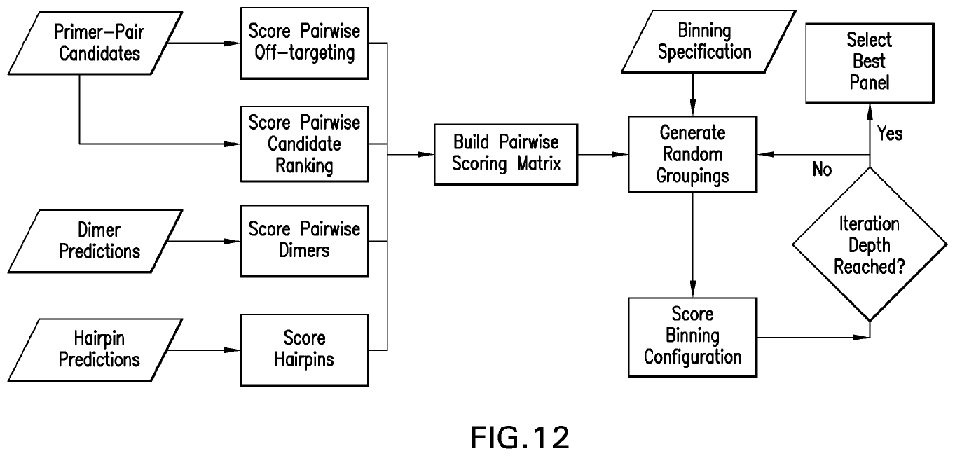

After individual primers are aligned, primer alignments on opposite strands (i.e. viable primer-primer amplification) under a user-specified length criteria (default: 500 bp) are aggregated for downstream analysis as potential off-target products. For each potential off-target product, hamming distance on the 3′ end of the primer binding site is calculated and used as a score to filter unlikely amplification products. This workflow is shown in FIG. 11 In addition to in silico PCR for predicting amplification products, a concurrent in silico PCR step is performed to predict all potential primer-primer interactions that could result in dimerization or hairpin formation. For this step, all interactions across primer pair candidates are constructed and passed through PRIMER3 to calculate complementarity and likelihood for dimerization. Similarly, PRIMER3 is also used to calculate the likelihood for self-binding and hairpin formation. Finally, independent thresholds are applied to the set of dimer and hairpin predictions (default: 25 & 40 respectively) and filtered results are aggregated for downstream analysis. This workflow is shown in FIG. 12.

To generate final panel designs, a scoring system was developed to rank combinations of primers using design annotations and predicted off-targeting. The scoring function considers 1) the number of predicted off-target products in a pool, 2) the number of dimer and hairpin products in a pool, and 3) the Primer3 ranks for individual candidate primers in a pool. An equation representing this scoring is as follows:

Sₚₒₒₗ=Wₒ* N_{offtarget}+W_{d}*N_{dimer}+Wₕ*N₊ₕₐᵢᵣₚᵢₙWᵣ* ΣCᵣₐₙₖ

In short, the score for a pool Sₚₒₒₗ is equal to the sum of the number of predicted off-target products in a pool N_{offtarget}, the number of predicted dimerization products N_{dimer}, the number of predicted hairpin products Nₕₐᵢᵣₚ, and the sum of all candidate ranks for primers in the pool Cᵣₐₙₖ. Each term has a weight applied that allows users to tailor designs for increased sensitivity or specificity (defaults: W₀=1, W_{d}=1, Wₕ=0.25, Wₜ=0.5).

Using this scoring system, final designs are produced by generating and scoring random combinations of candidate pairings that satisfy user-specified (binning) criteria about how many markers can be analyzed together via qPCR. After many iterations (default: e.g. 1×10⁶) are tested, the program outputs a final panel design with primer candidates organized into qPCR bins that can be validated downstream in the in vitro stages of the workflow (e.g., via amplicon fragment analysis and NGS).

### In Vitro Stages

A first lab-based PCR primer screening step is selected for its ability to be run quickly and cheaply. All that is needed are primers, which can be ordered and tested within 24 hours. No expensive/slow-to-make probes are required. Thus, roughly half of all primers identified by PrimerX can be eliminated without further stage testing.

### Exemplary Reaction Conditions Per Well are

10× AmpliTaq™ PCR buffer (Thermo): 1×
AmpliTaq Gold (5 U/uL, Thermo): 0.6 uL
dNTP mix (Sigma), 10 mM: 200 uM ea final
EvaGreen® (Biotium): 0.35× final
ROX Dye: 0.25 uM final
10× CutSmart® (New England Biolabs) Buffer (with 100 mM MgCl2): 4 mM MgCl12 final
Primers: 200 nM each
Genomic DNA: 10 ng total
Water: Up to 30 uL
   Cycling conditions:
95 °C for 10 min
95 °C for 15 seconds, 60 °C for 60 seconds, 45 total cycles. Read on 60 °C step, SYBR filter.
1.6 °C/s ramp rates

As with other dyes, EvaGreen also provides melt curve information, which provides a means of detecting off-targeting events without additional steps (see details below). The screening template is gDNA.

### Exemplary Primer Down-Selection Criteria

Closest Ct values to an endogenous control marker (IR) within the sample, but not lower than 5 cycles from the control. Primers lower than 5 cycles tended to be the result of off-targeting.

Absence of secondary melt-curve peaks, defined as any peak exceeding 10% of the primary peak's height.

Primary melt curve Tm >80 °C. Lower values indicate the primary curve is due to an off-targeting event, as >50% GC content sequences (common to this assay) should produce >80 °C values.

Peaks >10 Ct from the IR control are ignored, while values <5 cycles from the control led to the primer pair being discarded.

### Stage Output

Top 4 primer pairs per target. If less than 4 primer pairs are considered viable, the next best primers are carried through to the next steps, such that 4 primers are always moved into the next stage. The exception was markers with no passing primers, which led to the marker being completely dropped.

Fragment analysis of the amplicons also provides a complementary check for off-targeting beyond the in vitro PCR. The Agilent Fragment Analyzer system is a suitable platform, but similar platforms may also be used. Primers are ranked based upon the ratio of on-target to off-target bands (as the predicted size of the on-target bands was known). Primer pair candidates with off-targeting bands exceeding the primary band (quantifiable with area-under-the-curve densitometry measurements) are completely excluded from further testing. Remaining/surviving primers are ranked based upon cleanliness and used as tiebreakers for assessing the in vitro PCR results.

A notable finding from the in vitro stages of the workflow was a substantial gain in efficiency at the multiplex stage obtained from clean primer designs. Doing so appears to significantly lower the probability of issues when multiplexing loci together, and in turn, provides flexibility when creating groups.

Following the initial in vitro single plex PCR stage, a FAM (6-Carboxyfluorescein) target/JOE (5′-Dichloro-dimethoxy-fluorescein) Control duplex screening is performed, with a desired internal control primer pair as the second part of the duplex. As this internal control is to be used at the multiplex stage, the duplex PCR stage avoids any potential interactions with the control primers. FAM probes were selected for their simple conjugation chemistry for oligo manufacture.

### Exemplary Reaction Conditions Per Well ARE

10× AmpliTaq PCR buffer (Thermo): 1×
AmpliTaq Gold (5 U/uL, Thermo): 0.6 uL
dNTP mix (Sigma), 10 mM: 200 uM ea final
10× rCutSmart Buffer (with 100 mM MgC12, NEB): 4 mM MgC12 final
Buffy coat DNA: 10 ng total
Primers: 200 nM ea
Probes: 400 nM for targets, 200 nM for IR
Water: Up to 30 uL
   Cycling conditions (Same as EvaGreen Step)
95 C for 10 min
95 C for 15 seconds, 60 C for 60 seconds, 45 total cycles. Read on 60 C step, SYBR filter.
1.6C/s ramp rates

### Stage Output

Top 2 primer pairs per locus, selected based upon 1) Lowest Ct values and 2) Highest peak signal at cycle 45 (end of the run). This later criterion is used to select between otherwise identical (Ct value) primers. The best probe can also be selected at this stage, should multiple probes have been ordered per target.

### Secondi Silico Stage

This stage defines the final groups (bins) for multiplex PCR reactions. As discussed in the initial PrimerX section, primer pairs were designed only to multiplex with IR (present in all reactions), and not with pairs for the other targets. While this comes with the potential disadvantage of allowing incompatible (during eventual multiplexing) primers to pass early filtering stages, the issue is moot as 1) “clean” primers are much less susceptible to binning problems; and 2) the pipeline is designed to screen large groups of loci. PrimerX can easily avoid placing incompatible targets together due to the sheer number of possible bins.

Re-binning involves submitting the best 1-2 primer pairs per target (for all desired targets) into PrimerX, as identified in the preceding in silico stage. New primers are not generated; rather, the input primer pairs for all targets are checked against each other and the best pair per locus is placed into a bin of a defined size, using the same scoring equation used in the preceding in silico stage. Output: The best primer pair per target, and bin/well assignment for all targets.

Final In Vitro Stage—Dark Cycling

Dark cycling, as described herein, refers to a means of running pseudo-multiplexes without having different colors for each target available in order to reduce waiting time for probe manufacture. Multiplexing several targets on the same color would be impossible in conventional qPCR, as an operator couldn't decern which target produced the signal. To bypass this limitation, dark cycling places each target probe (or only a few target probes) into separate wells, while retaining all primer pairs for all targets. This allows the applicable probe to generate signal, while primer pairs without probes in that well amplify as they normally would but fail to produce signal. These “dark” primers can contribute to off-targeting of the measured signal, and by keeping them present/active, off-targeting can be measured without a true full color multiplex.

An endogenous control (IR, primers/probes) is present in all wells and uses another color (e.g., using JOE as the label). This serves as a reference point to judge a FAM signal from the probe that is present. The control itself isn't subjected to dark cycling, as there is no color collision with the actual target(s) being probed. Thus, dark cycling allows the artisan to assess the multiplex while more complex chemistries are synthesized over the longer time period required to do so. Thus, when full color materials arrive, problematic multiplexes have already been identified and (ideally) repaired. Exemplary run conditions can be those used in the earlier FAM-target/JOE-control duplex screen, but with primers for all targets present. The result is a conformation that the selected multiplex is successful.

### EXAMPLES

### Example 1

More than 100 GC-rich lung cancer markers were previously identified via a proprietary EpiCheck® NGS pipeline. These markers were analyzed by bespoke software (PrimerX) to predict optimal qPCR primer & probe candidates, using best practices combined with EpiCheck-specific requirements, such as inclusion of methylation-sensitive restriction enzyme (MSRE) cut sites. All in-vitro testing was performed on cfDNA and genomic DNA. For all markers, eight primer pairs were screened for performance and off-targeting using EvaGreen qPCR (QuantStudio 7 Pro), as well as direct product sizing (Fragment Analyzer). Top candidates (n=4) for each target were assessed for probe-based qPCR performance. The best two primers and probes for each target were binned in-silico by PrimerX into 3-, 4-, or 5-plex qPCR reactions and tested using dark cycling coupled with NGS for confirmation (or iterative redesign). Final multiplex reactions incorporated up to 5 fluorophores and were validated using clinical and analytical samples.

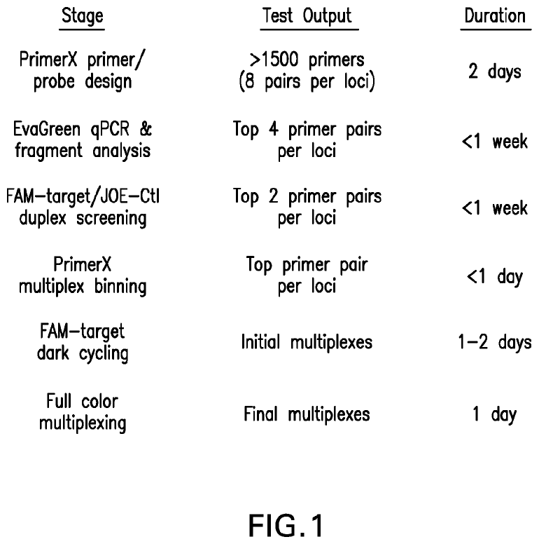

A flow diagram of the procedure is shown in FIG. 1. Workflow steps emphasize speed and performance by iteratively filtering out nonfunctional primers/probes through a combination of in-silico and lab-based stages. Total run time was 2-4 weeks, with screening content roughly halving at each stage. FAM probes are heavily emphasized throughout due to their rapid production rate, allowing for significant primer down-selection while awaiting other probe chemistries (e.g. ROX, ATTO, etc.). Optional iterative NGS can further be incorporated throughout the later half of the workflow.

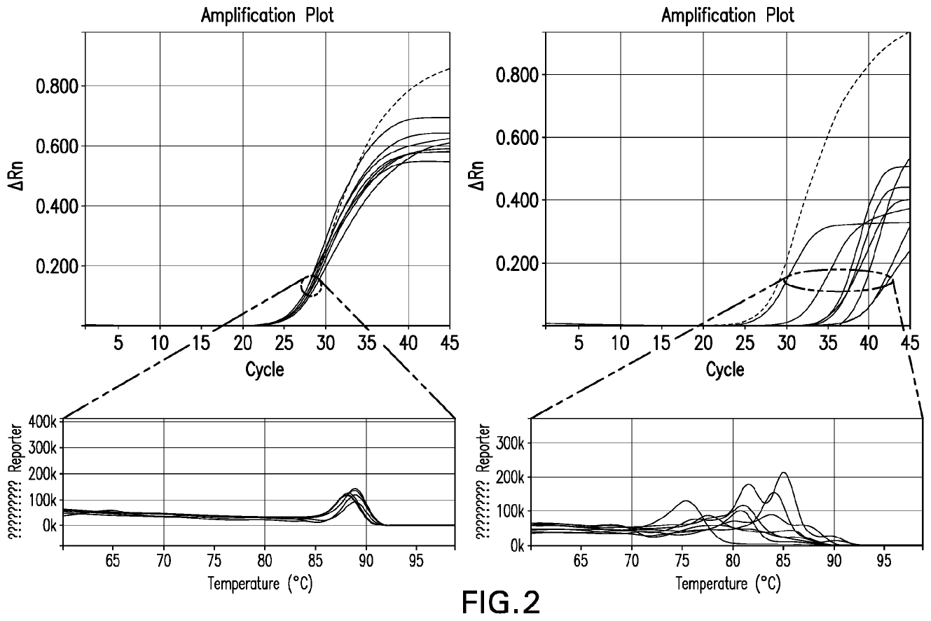

PrimerX utilizes a multi-stage pipeline for designing optimal primer pairs and probes. Candidates are initially generated through calls to PRIMER3, followed by a total output dimer crosscheck and genome alignment using BWA (Li and Durbin (2009), Bioinformatics 25:1754-60. Products are then scored by PrimerX for both individual and multiplex pairing potential. In turn, lab-refined singleplex outputs can serve as second round inputs, with added emphasis placed upon the multiplex binning stage. Eight primer pairs per target can be rapidly assessed for performance via EvaGreen qPCR and associated melt curve analysis. Examples of high (left) and low (right) functioning amplicons are shown, in reference to an internal control in FIG. 2.

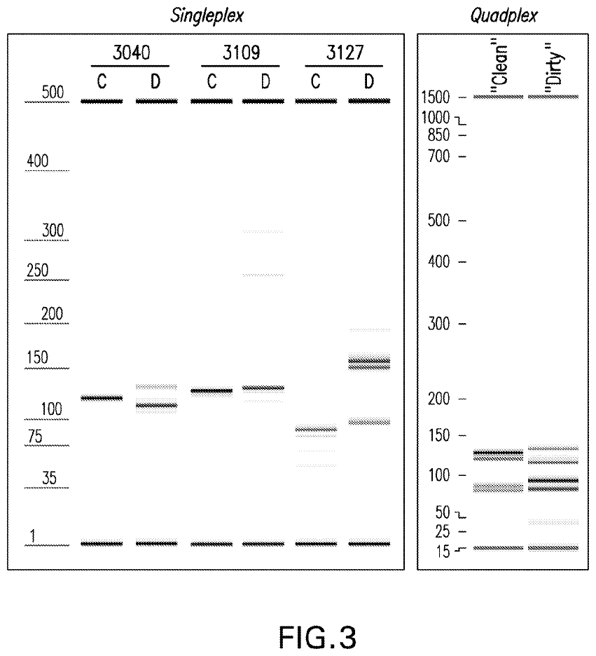

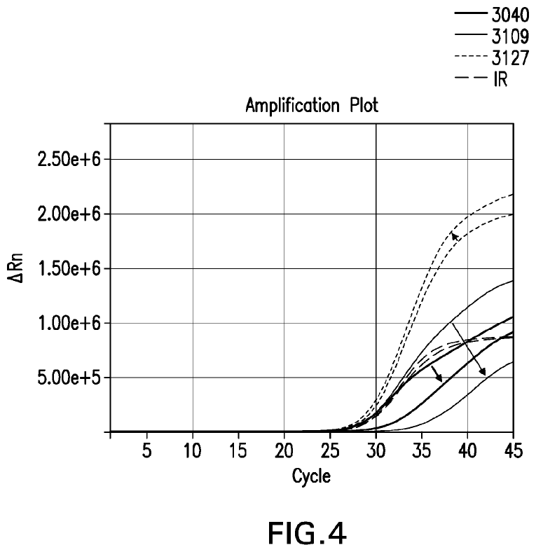

Eight primer pairs per target were screened in vitro with using a Fragment Analyzer System (Agilent) to visualize the amplicons produced, with the top 4 candidates progressing to subsequent stages based upon EvaGreen® (Biotium) Ct values and visualized/quantified on-target amplicons. Examples of “clean” (C) and “dirty” a(D) amplicons are shown (singleplex primer reactions), as well as resultant combinatorial multiplexes (all clean or all dirty pairs per group) in FIG. 3. qPCR output of “clean” and “dirty” quadplex groups were run as full color multiplexes, with results from both groups overlain in FIG. 4. Arrows indicate shifts from the “clean” to the “dirty” group. Although the “dirty” quadplex does not exhibit significant off-targeting, Ct & plateau heights were significantly affected during multiplex qPCR. Comparable results were observed in an additional 8 clean vs. dirty test groups (not shown). IR: Internal reference.

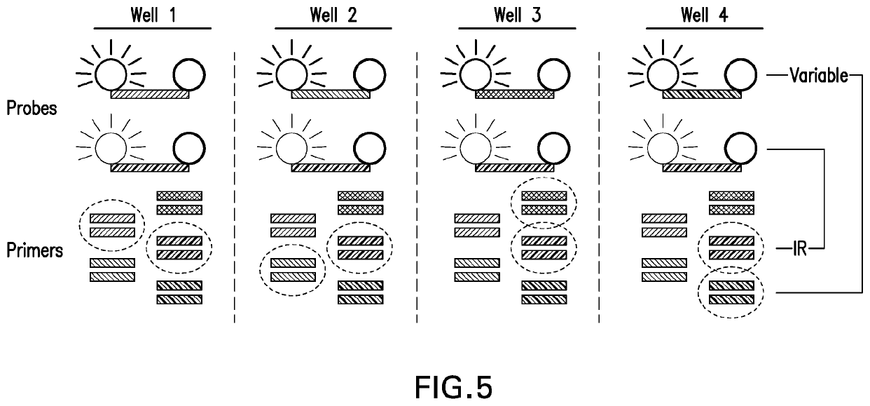

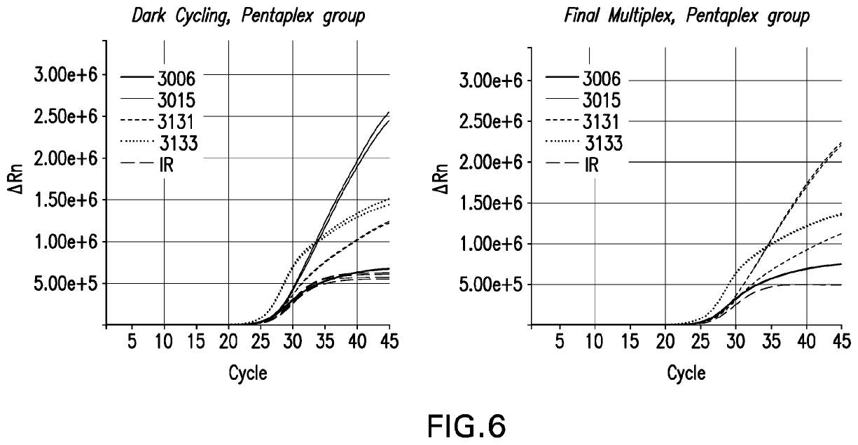

Full multiplex performance was mimicked by adding all intended primers to a reaction, but only one relevant probe per well, a procedure referred to herein as “dark cycling.” This is depicted schematically in FIG. 5. In each well, the “test probe relevant” primers are circled. Multiple wells (one per assessed target/locus) are then overlayed to simulate concurrent target amplification. This approach allows FAM-only probes to be tested while more complex chemistries are synthesized. IR: Internal reference. As shown in FIG. 6, dark cycling (left) closely mimics eventual multiplexes (right), allowing incompatibility issues to be addressed without the need to generate additional color channels for a full color multiplex. Minor deviations between dark cycling and full color multiplexing seen in this figure result from bleed-over effects and well to well variability.

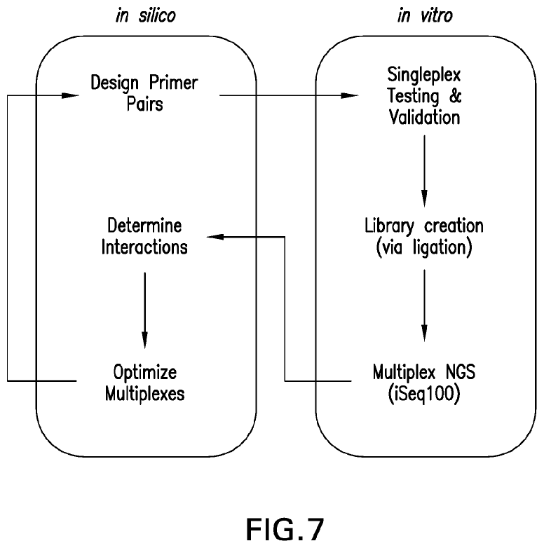

Primers produced by the methods described herein can be subjected to shallow sequencing to determine real-world off-targeting. Results can then be incorporated by PrimerX to inform binning. This is shown schematically in FIG. 7.

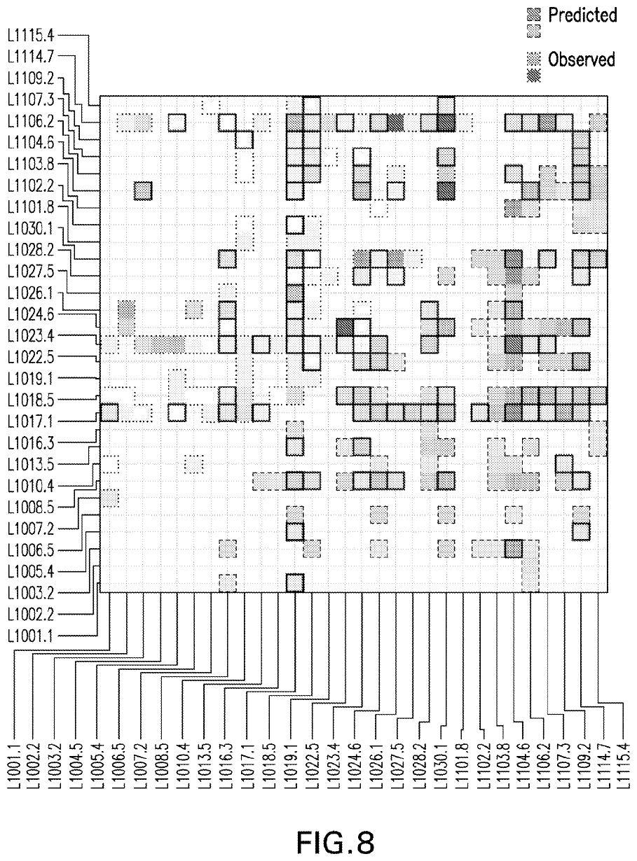

Thirty markers were run in multiplex on an iSeq 100 (Illumina) to determine off-targeting (dimers & unwanted amplicons), with the results shown in FIG. 8. Off-targets predicted by PrimerX are shown in blue, observed off-targets in red, and agreement between branches in outlined black. Scores from predicted and observed off-targeting were found to be significantly associated using a Spearman correlation significance test (p=0.03). Interactions found via NGS were subsequently analyzed by PrimerX to improve binning combinations.

Creation of large-scale qPCR multiplexes from NGS data is typically hampered by time-consuming identification of appropriate primers and their compatible multiplexes. The methods and compositions described herein, which combine an in silico and lab-based workflow, dramatically increase the efficiency of multiplex PCR projects by emphasizing low off-targeting singleplex primers, FAM-based dark cycling, with iterative NGS further accelerating the design process.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patent applications, patents, publications and other references mentioned in the specification are indicative of the levels of those of ordinary skill in the art to which the invention pertains and are each incorporated herein by reference. The references cited herein are not admitted to be prior art to the claimed invention.

Unless otherwise defined, all technical and scientific terms used herein have the same meaning as commonly understood by one of ordinary skill in the art to which this invention belongs. In the case of conflict, the present specification, including definitions, will control.

The use of the articles “a”, “an”, and “the” in both the description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about”, “approximately”, or “approximate”, when used in connection with a numerical value, means that a collection or range of values is included. For example, “about X” includes a range of values that are ±20%, ±10%, +5%, ±2%, ±1%, ±0.5%, ±0.2%, or ±0.1% of X, where X is a numerical value. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the terms “between X and Y” and “range from X to Y, are inclusive of X and Y and the integers there between. On the other hand, when a series of individual values are referred to in the disclosure, any range including any of the two individual values as the two end points is also conceived in this disclosure. For example, the expression “a dose of about 100 mg, 200 mg, or 400 mg” can also mean “a dose ranging from 100 to 200 mg”, “a dose ranging from 200 to 400 mg”, or “a dose ranging from 100 to 400 mg”.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for primer selection for a multiplex PCR reaction of a plurality of DNA templates, comprising:
(a) for each DNA template in the plurality of DNA templates, performing an in silico identification of a plurality of test primer pairs, and for each test primer pair calculating in silico pair a first scoring function value based on a set of predetermined selection criteria that comprise one or more of, and preferably each of
(i) a calculated ranking derived from a calculated melting point (Tm) range, a calculated primer length range, a calculated primer GC content and/or location value, and a calculated amplicon length,
(ii) a calculated number of predicted primer dimers within the test primer pair and between the test primer pair a control primer pair configured to amplify a predetermined control DNA template to be included in the multiplex PCR reaction,
(iii) a calculated overlap with a region of the DNA template that is a repeat of 10 bases or longer, and
(iv) a calculated number of off-target amplification products from a reference genome,
and for each DNA template in the plurality of DNA templates, using the first scoring function value to select from the plurality of test primer pairs a plurality of initial primer pairs;
(b) for each template in the plurality of DNA templates, screening each primer pair in the plurality of initial primer pairs in vitro in a single plex real time PCR reaction, calculating for each initial primer pair a second scoring function value based on set of selection criteria that comprise one or more of, and preferably each of, a cycle threshold (Ct) value, a melt curve peak threshold, a secondary melt curve peak, and a ratio of on-target to off-target amplicons produced, and for each DNA template in the plurality of DNA templates, using the second scoring function value to select from the plurality of initial primer pairs a plurality of intermediate primer pairs;
(c) for each template in the plurality of DNA templates, screening each primer pair in the plurality of intermediate primer pairs in vitro in a duplex real time PCR reaction with the control DNA template selected to be included in the multiplex PCR reaction and the control primer pair, and calculating for each intermediate primer pair a third scoring function value based on set of selection criteria that comprise one or more of, and preferably each of, a second Ct value and a peak signal at the end of the duplex real time PCR reaction, and for each DNA template in the plurality of DNA templates, using the third scoring function value to select from the plurality of intermediate primer pairs a plurality of candidate primer pairs;
(d) for each DNA template in the plurality of DNA templates, combinatorially binning the plurality of candidate primer pairs for each DNA template in silico to form a first primer multiplex and for each candidate primer pair calculating in silico pair a fourth scoring function value based on a set of predetermined selection criteria that comprise one or more of, and preferably each of
(i) a calculated ranking derived from a calculated melting point (Tm) range, a calculated primer length range, a calculated primer GC content and/or location value, and a calculated amplicon length,
(ii) a calculated number of predicted primer dimers within the candidate primer pair, between the candidate primer pair the control primer pair, and between the candidate primer pair all other primer pairs in the first primer multiplex,
(iii) a calculated overlap with a template region that is a repeat of 10 bases or longer,
(iv) a calculated number of off-target amplification products from a reference genome, and
and for each DNA template in the plurality of DNA templates, using the fourth scoring function value to select from the plurality of candidate primer pairs a plurality of final primer pairs;
(e) binning the final primer pair for each template in the plurality of DNA templates to form a set of multiplex primers; and for each template in the plurality of DNA templates, dark cycling the final primer pair in vitro to obtain a final Ct value, wherein the final primer pair is determined to be functional based on comparison of the final Ct value and peak signal obtained from the dark cycling of the final primer pair to the second Ct value and peak signal obtained in (c) for the final primer pair; and
(f) providing a pooled set of final primer pairs for the plurality of DNA templates.

2. The method of claim 1, wherein the plurality of DNA templates comprises at least 100 unique DNA templates.

3. The method of claim 1 or 2, wherein the number of test primer pairs for each DNA template in the plurality of DNA templates is at least 8.

4. The method of one of claims 1-3, wherein the first scoring function is calculated according to the following equation
Sₚₒₒₗ=Wₒ*N_{offtarget}+W_{d}*N_{dimer}+Wₕ+Nₕₐᵢᵣₚᵢₙ+Wᵣ*ΣCᵣₐₙₖ,
wherein
N_{offtarget} is the number of predicted offtarget products, N_{dimer} is the number of predicted dimer products, Nₕₐᵢᵣₚᵢₙ is the number of predicted hairpin products, Cᵣₐₙₖ is the calculated ranking in (a)(i), Wo is the relative weight applied to N_{offtarget}, W_{d} is the relative weight applied to N_{dimer}, Wₕ is the relative weight applied to Nₕₐᵢᵣₚᵢₙ, and Wᵣ is the relative weight applied to Cᵣₐₙₖ.

5. The method of one of claims 1-4, wherein the number of initial primer pairs for each DNA template in the plurality of DNA templates is at least 4.

6. The method of one of claims 1-5, wherein the second scoring function 7. The method of one of claims 1-6, wherein the number of intermediate primer pairs for each DNA template in the plurality of DNA templates is at least 4.

8. The method of one of claims 1-7, wherein the third scoring function is calculated by sorting each primer pair for the DNA template at a first level according to the second Ct value, and then sorting at a second level by peak signal height at a predetermined cycle number.

9. The method of one of claims 1-8, wherein the number of candidate primer pairs for each DNA template in the plurality of DNA templates is at least 2.

10. The method of one of claims 1-9, wherein the first scoring function is calculated according to the following equation
Sₚₒₒₗ=Wₒ*N_{offtarget}+W_{d}*N_{dimer}+Wₕ+Nₕₐᵢᵣₚᵢₙ+Wᵣ*ΣCᵣₐₙₖ,wherein
N_{offtarget} is the number of predicted offtarget products, N_{dimer} is the number of predicted dimer products, Nₕₐᵢᵣₚᵢₙ is the number of predicted hairpin products, Cᵣₐₙₖ is the calculated ranking in (d)(i), Wo is the relative weight applied to N_{offtarget}, W_{d} is the relative weight applied to N_{dimer}, Wₕ is the relative weight applied to Nₕₐᵢᵣₚᵢₙ, and Wᵣ is the relative weight applied to Cᵣₐₙₖ.

11. The method of one of claims 1-10, wherein the final primer pair is identified as functional when the final Ct value for the final primer pair is within 1.5 cycles of the second Ct value for the final primer pair, and when the peak signal obtained from the dark cycling of the final primer pair is equal to or greater than the peak signal obtained in (c) for the final primer pair.

12. A method for performing a multiplex PCR reaction of a plurality of DNA templates, comprising:
obtaining a nucleic acid sample to be amplified;
preparing a PCR reaction mixture comprising the nucleic acid sample and the pooled set of final primer pairs for the plurality of DNA templates according to one of claims 1-11; and
incubating the PCR reaction mixture under conditions sufficient to amplify the plurality of DNA templates present in the nucleic acid sample.

13. The method of claim 12, wherein the nucleic acid sample comprises or consists of cell free DNA (cfDNA).
